(19) United States
(12) Patent Application Publication
(10) Pub. No.: US 2026/0113526 A1
(43) Pub. Date: Apr. 23, 2026

(54) Image Acquisition Assembly and Holding Device for a Prism and at Least Two Image Sensors

(71) Applicant: KARL STORZ SE & Co. KG, Tuttlingen (DE)

(72) Inventors: Andreas Frick, Donaueschingen (DE); Lars Hedemann, Rudersberg (DE)

(73) Assignee: KARL STORZ SE & Co. KG, Tuttlingen (DE)

(21) Appl. No.: 19/363,332

(22) Filed: Oct. 20, 2025

(30) Foreign Application Priority Data
Oct. 21, 2024 (DE) 10 2024 130 623.8

(51) Int. Cl. H04N 23/54; G03B 33/12; H04N 23/50

(52) U.S. Cl. CPC H04N 23/54; G03B 33/12; H04N 23/555

(57) ABSTRACT

An image acquisition assembly for an optical instrument with a beam-splitting prism having a light entry surface, a dichroic reflective interface, a first light exit surface and a second light exit surface, at least two image sensors, and a holding device. A first image sensor is on the first light exit surface, and a second image sensor is on the second light exit surface. The holding device includes a first mount connecting the first image sensor to the prism and a second mount connecting the second image sensor to the prism. The first mount includes a stop for bearing against a first stop surface of the prism and for bearing against a stop surface on the first image sensor. The second mount has a stop for bearing against a second stop surface of the prism and/or for bearing against a stop surface on the second image sensor.

### CROSS REFERENCE TO RELATED APPLICATIONS

The present invention claims priority to German Patent Application No. 10 2024 130 623.8, filed on Oct. 21, 2024, and entitled, “Bilderfassungsbaugruppe und Halteeinrichtung für ein Prisma und wenigstens zwei Bildsensoren,” the entire disclosure of which is incorporated herein by reference.

### TECHNICAL FIELD

The present invention relates to an image acquisition assembly comprising at least two image sensors on a beam-splitting prism and a holding device for mechanically rigidly holding the at least two image sensors on the prism, and to an optical instrument comprising such an image acquisition assembly.

### BACKGROUND

In medical engineering, but also for applications outside of medicine, increasingly smaller optical instruments are being developed, for example rigid or flexible endoscopes. The instruments often comprise multiple modules, such as an electronic module for generating and controlling light, a flexible or rigid shaft module for directing light beams to the examination site, and an image acquisition assembly for capturing optical images at the examination site. It is important to precisely arrange components of the individual modules and assemblies in order to allow for high-quality imaging and localization of images. DE 2019 106 453 A1, for example, describes an endoscope comprising an optical system at the end of an elongated shaft. The optical system comprises a prism, which is held in a prism holder, and an image converter for capturing images, which is arranged on a light exit surface of the prism. The prism holder is designed as a cylindrical sleeve and has multiple stops for orienting the prism in the prism holder and for positioning the prism in the optical system.

In many cases, it is desirable or necessary to acquire images in different wavelength ranges inside and outside the wavelength range visible to the healthy human eye. For example, one aim is to observe visible light and fluorescent light simultaneously.

If different image sensors are used for different wavelength ranges, the light must first be split into wavelength ranges—in particular by means of a dichroic reflective layer or interface—and then fed separately to different image sensors. The dichroic reflective layer or interface is usually provided in a prism that has one light entry surface and multiple light exit surfaces. One image sensor is arranged on each light exit surface in order to acquire images in the corresponding wavelength range.

US 2022/0179189 A1, for example, describes an endoscope for dual image acquisition. The image acquisition module of the endoscope comprises a beam-splitting prism for splitting a measurement beam in one direction toward a first image sensor and in another direction toward a second image sensor. The image sensors and the prism may be arranged, for example, directly in a housing of the endoscope or in a housing of an image acquisition assembly, such as in a camera head mounted on the endoscope housing. Miniaturizing an image acquisition assembly consisting of a prism and multiple image sensors is a technological and manufacturing challenge, with particular attention having to be paid to a precise geometric arrangement and orientation of the components in the assembly and to correct beam guidance.

### BRIEF DESCRIPTION OF THE DISCLOSURE

One object of the present invention is to provide an improved image acquisition assembly comprising a holding device for mechanically rigidly holding at least two image sensors on a beam-splitting prism, an improved holding device, and an improved optical instrument. In particular, the aim is to allow for reliable and precise positioning of the prism and image sensors relative to one another and relative to other components of the image acquisition assembly, easy installation of the prism and image sensors in the image acquisition assembly, and a reduction in the required installation space.

This object is achieved by an image acquisition assembly, a holding device for holding a prism and at least two image sensors of such an image acquisition assembly, and an optical instrument comprising such an image acquisition assembly, according to the independent claims.

Advantageous embodiments and developments of the invention can be found in the dependent claims.

An image acquisition assembly according to the invention comprises a beam-splitting prism having a light entry surface, a dichroic reflective layer or interface, at least a first light exit surface and a second light exit surface, at least two image sensors, and a holding device for holding the prism and the at least two image sensors. A first image sensor is arranged on the first light exit surface of the prism, and a second image sensor is arranged on the second light exit surface of the prism. The holding device comprises a first mount for mechanically rigidly connecting the first image sensor to the prism and a second mount for mechanically rigidly connecting the second image sensor to the prism. The first mount has at least one stop for bearing against a first stop surface of the prism and for bearing against a stop surface on the first image sensor. The second mount has at least one stop for bearing against a second stop surface of the prism and/or for bearing against a stop surface on the second image sensor.

The image acquisition assembly and the holding device according to the invention are in particular intended and designed as a component of an endoscope or of another optical instrument for microinvasive medical procedures. The image acquisition assembly comprising the holding device, the prism and the image sensors therefore has linear dimensions of no more than one centimeter or in the order of a few millimeters.

In particular, the image acquisition assembly comprises a prism which comprises two sub-prisms and a dichroic reflective layer or interface therebetween. The dichroic reflective layer or interface reflects light in a first spectral range and transmits light in a second spectral range, the first spectral range and the second spectral range being disjoint. For example, by means of the dichroic reflective layer or interface, light entering through the light entry surface in the spectral range visible to the healthy human eye is directed to the first image sensor, and infrared light is directed to the second image sensor. This can allow for simultaneous observation of the visible light reflected or remitted by an object and of the infrared light reflected or remitted by the object. It is also conceivable that the prism has a second dichroic reflective layer or interface and has three light exit surfaces. In this case, three image sensors and a holding device comprising three mounts with stops for the prism and/or image sensors are provided. Analogously, more than three light exit surfaces could also be provided on the prism, and correspondingly more than three image sensors could be provided.

The first image sensor and the second image sensor may each have a wavelength-dependent or wavelength-selective sensitivity. The first image sensor and the second image sensor may each comprise a filter or form an assembly with a filter that blocks or suppresses unwanted wavelengths.

The mounts of the holding device establish a mechanically rigid connection between the prism and the respective image sensor such that, once the prism and the image sensors have been installed in the mounts, any relative movement is preferably impossible. Following insertion, the mount, the prism and the image sensor are positioned relative to one another in a mechanically rigid manner by the stop such that these components are securely held in a predefined orientation relative to one another. The prism, the image sensors and the associated mounts may be rigidly fixed in their position relative to one another, for example by a form-fitting or materially bonded connection, by a connecting element such as a clamping element, a locking element or a spring element, or by other suitable types of connection. Following installation, there is preferably a permanent connection between the mounts and the prism and/or the image sensors.

The image acquisition assembly according to the invention allows precise positioning of the at least two image sensors and the prism relative to one another and independently of other assemblies or components of an optical instrument. The stop of the first mount allows an exact orientation of the first image sensor on the prism for optimal imaging using the light of the first spectral range, and the stop of the second mount allows an exact orientation of the second image sensor on the prism for optimal imaging using the light of the second spectral range. A parallelism of the light entry and exit surfaces between the prism and the image sensors can thus be ensured. Production of the image acquisition assembly is made efficient by the predefined stops of the holding device, and correct installation of the image sensors is ensured. Assembly of the optical instrument is also facilitated.

According to one variant of the image acquisition assembly, the first mount and the second mount are designed as separate components. In this case, the first mount and the second mount may be produced separately. Alternatively, the first mount and the second mount are produced at the same time and are separated from each other only at the end of joint production.

The use of two separate components allows for independent positioning of the two mounts and thus allows for particularly precise positioning of the image sensors on the light exit surfaces of the prism.

According to another variant of the image acquisition assembly, the first mount and the second mount are at least either directly mechanically rigidly connected or are provided as one piece.

The first mount and the second mount may be manufactured separately and thereafter directly mechanically rigidly and permanently connected to each other, for example in a materially bonded and/or form-fitting manner. The first mount and the second mount may each be originally manufactured in one piece or assembled from multiple components. The first mount and the second mount are connected, for example, by a welded or soldered connection, an adhesive connection, a crimped connection or another connection based on plastic deformation, and/or a screw connection. In particular, the separately manufactured mounts bear directly against each other and are spatially separated at most by a weld seam, a layer of solder, or a layer of adhesive, but are mechanically connected.

Alternatively, the holding device may originally be manufactured already in one piece.

The direct mechanically rigid connection of the mounts or the one-piece manufacture of the holding device can allow for a particularly robust design of the image acquisition assembly. Furthermore, the direct mechanically rigid connection of the mounts or the one-piece manufacture of the holding device can allow for a particularly efficient and thus also particularly cost-effective production of the image acquisition assembly.

According to one embodiment of an image acquisition assembly according to the invention, in the case of the holding device, a stop surface of the first mount is orthogonal to the first light exit surface of the prism and/or is orthogonal to a light entry surface of the first image sensor. Furthermore, a stop surface of the second mount may be arranged orthogonal to the second light exit surface of the prism and/or orthogonal to a light entry surface of the second image sensor. An orthogonal design of the stop surfaces of the two mounts aids a parallel arrangement of the light exit surfaces of the prism and the light entry surfaces of the image sensors.

In the context of this invention, two surfaces are to be considered orthogonal if a light beam hits the light exit or light entry surfaces at around 90 degrees such that substantially no reflection or diffraction occurs at the surfaces. In particular, two surfaces are to be considered orthogonal if the surface normals thereof enclose an angle between 70 degrees and 110 degrees, or an angle between 80 degrees and 100 degrees, or an angle between 85 degrees and 95 degrees, or an angle of exactly 90 degrees within the manufacturing tolerances.

According to one variant of the image acquisition assembly according to the invention, the stop of the first mount has a common stop surface for bearing against the first stop surface of the prism and against the stop surface of the first image sensor, and/or the stop of the second mount has a common stop surface for bearing against the second stop surface of the prism and/or against the stop surface of the second image sensor. A common stop surface for the prism and the first or second image sensor simplifies the design and manufacture of the holding device. Furthermore, the installation of the prism and image sensors on the holding device can be kept simple.

In the context of the invention, the mounts of the holding device may each have a single common stop surface for the prism and a light sensor, such as a step or edge that comes to lie against a corresponding stop surface on the prism or light sensor. A mount may also comprise multiple common stop surfaces, against which the prism and the image sensor bear, for example with different regions. In particular in the case of a one-piece holding device, common stop surfaces on the first and second mount may allow for easy insertion of the prism and image sensors. However, the mounts may each also have a stop in the form of a stop structure comprising multiple differently arranged or oriented stop surfaces, such as a first stop surface and a second stop surface, as described below. The multiple different stop surfaces are formed, for example, on different surfaces of the mount or in the manner of steps on one surface side of the mount. Multiple stop surfaces allow a differentiated adaptation of the holding device to the design of the image acquisition assembly.

In another variant of the image acquisition assembly according to the invention, the stop of the first mount has a first stop surface for bearing against the corresponding first stop surface of the prism and a second stop surface for bearing against the corresponding stop surface on the first image sensor. Furthermore, the stop of the second mount may have a first stop surface for bearing against the corresponding second stop surface of the prism and a second stop surface for bearing against the corresponding stop surface on the second image sensor. The first image sensor and the second image sensor can thus be arranged on the respective mounts independently of a stop of the prism on the holding device and can enter into a mechanically rigid connection with the prism. Advantageously, the first mount and the second mount are designed as two separate components and can be attached to the prism separately from each other. Alternatively, the holding device may be formed in one piece, as described above.

In this embodiment variant of the holding device, as described here, at least either the first stop surface of the first mount may be orthogonal to the intended orientation of the first light exit surface of the prism, or the second stop surface of the first mount may be orthogonal to the intended orientation of a light entry surface of the first image sensor. In particular, both the first stop surface and the second stop surface of the first mount may be orthogonal to the light entry and light exit surface, respectively.

Stop surfaces oriented in this way can allow for an exact lateral orientation of the first image sensor relative to the first light exit surface of the prism.

Furthermore, the first stop surface of the second mount may be orthogonal to the intended orientation of the second light exit surface of the prism, or the second stop surface of the second mount may be orthogonal to the intended orientation of a light entry surface of the second image sensor. Again, both the first stop surface and the second stop surface of the second mount may be orthogonal to the light exit and light entry surface, respectively.

Stop surfaces oriented in this way can allow for an exact lateral orientation of the second image sensor relative to the second light exit surface of the prism.

According to another embodiment of an image acquisition assembly according to the invention, in the case of the holding device, the stop of the first mount, in particular the first stop surface of the first mount, is intended to bear against a surface region of the prism that faces away from the light entry surface of the prism.

In particular, the surface region of the prism that faces away from the light entry surface of the prism is not parallel to the light entry surface and in particular does not adjoin the light entry surface. In particular, the second light exit surface of the prism is arranged between the light entry surface of the prism and the surface region of the prism against which the stop or the first stop surface of the first mount bears.

In particular, the surface normals of the light entry surface, the first light exit surface, the second light exit surface and the surface region of the prism that faces away from the light entry surface, against which the stop or the first stop surface of the first mount bears, lie in one plane.

Furthermore, in the case of the holding device, the stop of the second mount, in particular the first stop surface of the second mount, may be intended at least partially to bear against a surface region of the prism that comprises the light entry surface of the prism. In this case, the stop or the first stop surface of the second mount advantageously bears only partially against the surface region that comprises the light entry surface, and leaves another part free, in particular the sub-region at which an incident light beam hits the light entry surface.

According to yet another embodiment of an image acquisition assembly according to the invention, the stop of the first mount may have one stop surface for bearing against an edge region of the first light exit surface of the prism and another stop surface for bearing against an edge region of a light entry surface of the first image sensor.

Furthermore, the stop of the second mount may have one stop surface for bearing against an edge region of the second light exit surface of the prism and another stop surface for bearing against an edge region of a light entry surface of the second image sensor. The first stop surface of the second mount is in particular intended and designed to bear against an edge region of the light entry surface or of the planar surface region of the prism that comprises the light entry surface. Since a first stop surface of the first mount bears against a surface region that faces away from the light entry surface of the prism and the first stop surface of the second mount bears against a surface region of the prism that comprises the light entry surface of the prism, sufficient installation space can be available for both stops even when the prism and the mounts are very small in size. The prism therefore does not have to be made larger just to be able to implement the stops.

The stop surfaces for bearing against the edge regions of the prism and image sensors may be designed as a first stop surface and a second stop surface of the stops of the mounts. However, in addition to the above-described first stop surface and second stop surface, the stop of a mount may also provide a third stop surface and a fourth stop surface for this purpose, which bear against the edge regions. For example, the first stop surface and the second stop surface may form a common stop, for example oriented orthogonal to the light entry and exit surfaces, or may be provided as separate stop surfaces. The third stop surface and the fourth stop surface, which bear against the edge regions, extend parallel to the light entry and exit surfaces. In this exemplary embodiment, the stop of the mounts of the holding device is designed in the form of a stop structure comprising multiple different stop surfaces.

In summary, in a variant of the holding device as described here, the stop of the first mount may have a third stop surface for bearing against an edge region of the first light exit surface of the prism and a fourth stop surface for bearing against an edge region of a light entry surface of the first image sensor, and/or the stop of the second mount may have a third stop surface for bearing against an edge region of the second light exit surface of the prism and a fourth stop surface for bearing against an edge region of a light entry surface of the second image sensor.

Each mount may therefore have two, in particular parallel, surface regions which are each planar and face away from each other, which surface regions may bear in edge regions of the respective light exit surface of the prism and the light entry surface of the respective image sensor in order to allow for an exactly parallel orientation of the light entry surface of the image sensor relative to the light exit surface of the prism.

Since these stop surfaces only bear against an edge region of the light entry or exit surfaces, an interior region surrounded by the edge regions is free of stop surfaces of the mounts. In particular, an interior region of the light entry or exit surfaces can be left free, through which at least much of the light radiation enters and exits. As a result, the stop surfaces against the edge regions do not impair image acquisition by the image sensors. Advantageously, in the case of the first mount, a first gap between the prism and the first image sensor is defined by the stop surface for bearing against the edge region of the first light exit surface of the prism and the stop surface for bearing against the edge region of the light entry surface of the first image sensor. Analogously, in the case of the second mount, a second gap between the prism and the second image sensor is defined by the stop surface for bearing against the edge region of the second light exit surface of the prism and the stop surface for bearing against the edge region of the light entry surface of the second image sensor. The gaps are defined by the distance between the respective stop surfaces and thus determine the distance between the prism and the image sensor. The gaps advantageously serve as an air gap between the prism and the image sensor and thus form an optical layer that can be used to reflect and transmit incident light radiation. Depending on the angle of incidence of the light radiation within the prism onto one of the light exit surfaces or an optical layer adjoining the latter, the light radiation will be reflected or allowed to pass through to the respective image sensor. The geometry of the prism and the dichroic reflective layer or interface provided therein serves to deflect the sub-beams to the respective light exit surfaces and thus to the light sensors.

In the case of a holding device having stop surfaces for bearing against edge regions of the light entry and exit surfaces, as described above, advantageously the first mount may comprise a film component having a first planar surface region and a second planar surface region, wherein the first planar surface region and the second planar surface region of the film component are parallel and face away from each other. The first planar surface region of the film component may form the stop surface for bearing against an edge region of the first light exit surface of the prism, and the second planar surface region of the film component may form the stop surface for bearing against an edge region of a light entry surface of the first image sensor. The thickness of the film component thus determines the size of the gap between the prism and the image sensor.

In particular, the film component is made of a high-precision measurement film, which has two exactly planar and parallel surfaces facing away from each other and thus has a constant thickness. Films can be produced in almost any thickness and are available on the market in many different thicknesses. A suitably thin film component can allow for a very small distance between the light entry surface of the first image sensor and the first light exit surface of the prism. Advantageously, a film having a thickness in the micrometer range or less can be used.

Furthermore, in one embodiment variant of the holding device, for example, the stop surface for bearing against an edge region of the first light exit surface of the prism and the stop surface for bearing against an edge region of a light entry surface of the first image sensor of the first mount, as described above, may each be U-shaped or C-shaped. As an alternative or in addition, the stop surface for bearing against an edge region of the second light exit surface of the prism and the stop surface for bearing against an edge region of a light entry surface of the second image sensor of the second mount, as described above, may each be U-shaped or C-shaped. U-shaped or C-shaped stop surfaces can bear against three of four sides or edges of the surfaces and can prevent the image sensor from tilting relative to the prism.

Alternatively, in the case of the holding device, the stop surfaces for bearing against edge regions of the light entry and exit surfaces may each be annular. Annular stop surfaces can bear against all four sides or edges of the surfaces and can further prevent the image sensor from tilting relative to the prism. Furthermore, the U-shaped, C-shaped or annular stop surfaces of the mounts aid an exact parallel arrangement of the sensor entry surfaces and prism exit surfaces relative to one another. By selecting the shape of the stop surface, it is also possible to determine the shape of the interior region of the prism that remains free of stop surfaces and thus corresponds to the shape of the gap or the area of the air gap. For example, U-shaped or C-shaped stop surfaces can create a larger gap area than annular stop surfaces, and thus can form a larger optical layer. Advantageously, the stop structure of the first mount is designed such that the light radiation entering the prism hits the first gap at an angle at which the light radiation is reflected as completely as possible by the dichroic reflective layer or interface.

In one advantageous embodiment of an image acquisition assembly according to the invention, a beam-splitting prism is provided with a dichroic reflective layer or interface which is arranged in or parallel to an angle bisector plane of the first light exit surface and the second light exit surface of the beam-splitting prism. For example, a beam-splitting prism may be provided, the light entry surface and the first light exit surface of which form an angle of 45 degrees, and the light entry surface and the second light exit surface of which form an angle of 90 degrees, and which has a dichroic reflective layer or interface that forms an angle of 22.5 degrees in each case with the first light exit surface and with the second light exit surface. The first mount is advantageously U-shaped or C-shaped with an open side oriented toward the light entry surface of the prism, in order to form a large-area reflection gap. The second mount advantageously has an annular topology, which allows a sub-beam coming from the interface to pass through to the second light sensor as completely as possible and at the same time aids a precise orientation of the sensor on the prism.

This embodiment is particularly suitable for very small image acquisition assemblies, such as those used in endoscopes for example, since the geometry of the prism requires little installation space and the design of the holding device ensures that the image sensors are parallel.

Especially when the light entry surface of the prism is intended to be arranged orthogonal to the longitudinal axis of a thin shaft, the U-shaped or C-shaped design of the first mount can reduce the required installation space and thus allow for a more favorable ratio between the size of the prism and the image sensors on the one hand and the size of the cross section of the shaft on the other hand. In contrast, the second mount can have an annular topology even when the installation space is very limited.

In yet another embodiment of an image acquisition assembly according to the invention, the first mount may at least partially surround the prism on at least three different planar or substantially planar surface regions that adjoin the first light exit surface. In particular, the first mount surrounds the prism insofar as the first mount has surface regions which are each at least partially arranged opposite one of at least three planar surface regions of the prism that adjoin the first light exit surface. Furthermore, the second mount may at least partially surround the prism on at least three planar or substantially planar surface regions that adjoin the second light exit surface. In particular, the second mount surrounds the prism insofar as the second mount has surface regions which are each at least partially arranged opposite one of at least three planar surface regions of the prism that adjoin the second light exit surface.

The fact that a mount at least partially surrounds the prism can facilitate or improve both the orientation of the mount relative to the prism and also the mechanically rigid and permanent connection of the mount to the prism.

Each of said surface regions of a mount may bear against the opposite surface region and/or may enter into a mechanically rigid connection therewith, for example by means of an adhesive connection.

In one embodiment variant of the image acquisition assembly, for example, the first mount comprises at least three frame portions, which in the intended arrangement are each arranged opposite one of three different planar or substantially planar surface regions of the prism that adjoin the first light exit surface of the prism, and/or the second mount comprises at least three frame portions, which in the intended arrangement are each arranged opposite one of three different planar or substantially planar surface regions of the prism that adjoin the second light exit surface of the prism.

In particular, each frame portion of a mount may have one or more regions located opposite an associated planar surface region of the prism that adjoins one of the light exit surfaces of the prism.

Each frame portion of a mount may bear against the opposite surface region and/or enter into a mechanically rigid connection therewith, for example by means of an adhesive connection.

Said frame portions of the mounts may facilitate or improve both the orientation thereof relative to the prism and also the mechanically rigid and permanent connection thereof to the prism.

In a further embodiment of an image acquisition assembly according to the invention, use can advantageously be made of a beam-splitting prism comprising a first sub-prism and a second sub-prism, between which the dichroic reflective layer or interface is arranged. In this case, for example, either the first mount may be intended and designed to be directly mechanically connected only to the first sub-prism, and/or the second mount may be intended and designed to be directly mechanically connected only to the second sub-prism. Analogously, in the case of a prism comprising three sub-prisms, a third mount may be provided, which is directly mechanically connected only to the third sub-prism. With such a design of the prism and the holding device, the image acquisition assembly can be flexibly adapted to different requirements of an optical instrument; for example, prisms with different optical characteristics can easily be used, and holding devices adapted therefor can be kept ready.

Alternatively, for example, the first mount may be intended and designed to be directly mechanically connected both to the first sub-prism and to the second sub-prism, and/or the second mount may be intended and designed to be directly mechanically connected both to the first sub-prism and to the second sub-prism. With a holding device designed in this way, advantageously the sub-prisms can also be oriented relative to each other.

In one variant of the image acquisition assembly, the plane in which the dichroic reflective layer or interface is arranged does not intersect at least either the first mount or the second mount, or intersects it only in a region facing away from the light entry surface of the beam-splitting prism. In particular, the plane in which the dichroic reflective layer or interface is arranged intersects neither a frame portion of the first mount nor a frame portion of the second mount. This design is particularly useful for separate mounts, in order to be able to address specifics of sub-regions above and below the interface of the prism separately.

In one embodiment of an image acquisition assembly according to the invention, the holding device advantageously comprises at least one through-opening for applying adhesive or solder in order to connect the first mount to the prism and/or the second mount to the prism in a materially bonded manner.

In particular, the at least one through-opening is arranged in a frame portion of the first mount and/or the second mount and allows the application of adhesive or solder to a planar surface region of the prism that adjoins the first light exit surface and/or the second light exit surface of the prism. Each mount may have multiple through-openings, which in particular may be arranged symmetrically.

According to another aspect of the present invention, an optical instrument is provided, comprising an image acquisition assembly as described herein.

In one embodiment variant, the optical instrument comprises a shaft having a distal end, wherein the image acquisition assembly is arranged at the distal end or in the region of the distal end of the shaft. In particular, the image acquisition assembly is arranged in the shaft. Advantageously, the image acquisition assembly can be inserted into the shaft and removed therefrom. The shaft may be rigid and straight or curved or partially or completely flexible. The shaft serves in particular to guide light and electrical leads to and from the image acquisition assembly, as is customary in the case of image acquisition instruments.

In particular, the optical instrument is provided as an endoscopic device. An “endoscopic device” is intended to mean, in particular, a preferably functional component, in particular a subassembly and/or a structural and/or functional component of an endoscopic instrument and/or of an endoscope. Alternatively, the endoscopic device may form an endoscope and/or an endoscopic instrument, at least in part, preferably at least to a large extent, and particularly preferably in its entirety. In particular, the term “endoscopic” is also intended to mean minimally invasive. For example, the endoscopic device is configured to be inserted at least in part, and preferably at least to a large extent, into an artificial and/or natural opening, in particular an opening in the body, in order to carry out a treatment and/or examination there. For example, an endoscopic instrument may also be an endoscopic forceps instrument, an endoscopic scissors instrument, an endoscopic scalpel instrument, an endoscopic clamp instrument, or the like.

It is emphasized that the image acquisition assembly and the holding device according to the invention are also suitable for optical instruments, in particular endoscopes, comprising more than two image sensors and for prisms comprising more than two light exit surfaces. In this case, the holding device may have further mounts; for example, a separate mount for each image sensor for connection to a light exit surface of the prism. However, it is also possible for more than one image sensor in the same mount to be rigidly connected to the same light exit surface of the prism.

### BRIEF DESCRIPTION OF THE DRAWINGS

Further advantages will become apparent from the following description of the drawings. The drawings show exemplary embodiments of the invention. The drawings, the description and the claims contain numerous technical features in combination. Advantageously, a person skilled in the art will also consider the features individually and combine them into useful further combinations or associate them with other exemplary embodiments of the invention, as described above.

FIG. 1 shows a schematic axonometric illustration of an optical instrument comprising one variant of an image acquisition assembly according to the invention.

FIG. 2 shows a schematic axonometric illustration of the image acquisition assembly of FIG. 1 comprising one embodiment of a holding device according to the invention.

FIG. 3 shows a schematic illustration of a section through the image acquisition assembly of FIG. 2.

FIG. 4 shows a schematic axonometric illustration of a first mount of the embodiment of the holding device of FIG. 2 in a first view.

FIG. 5 shows another schematic axonometric illustration of the first mount of FIG. 2 in a second view, and a first image sensor in an exploded view.

FIG. 6 shows a schematic axonometric illustration of a second mount of the embodiment of the holding device of FIG. 2 in a first view.

FIG. 7 shows another schematic axonometric illustration of the second mount of FIG. 2 in a second view, and a second image sensor in an exploded view.

FIG. 8 shows a schematic axonometric illustration of another variant of an image acquisition assembly according to the invention.

FIG. 9 shows a schematic axonometric illustration of yet another variant of an image acquisition assembly according to the invention.

### DETAILED DESCRIPTION OF THE VARIOUS EMBODIMENTS

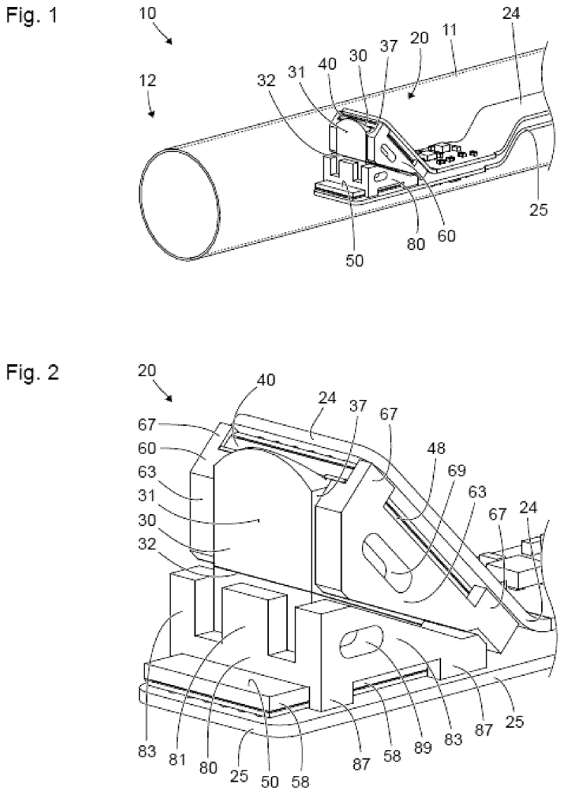

FIG. 1 contains a schematic axonometric illustration of part of an optical instrument 10, namely an endoscope comprising a shaft 11. The endoscope 10 may be intended and designed for medical or technical tasks. The shaft 11 may be rigid and straight or curved or partially or completely flexible. The shaft 11 can be connected in a known manner to other assemblies of the endoscope 10, for example to a light-generating assembly, an optical assembly, a control assembly, an operating unit, etc. By way of example, one embodiment of an image acquisition assembly 20 comprising a holding device 60, 80 according to the present invention is accommodated in the shaft 11 of the endoscope 10, as will be explained in detail below with reference to FIG. 2 to 7. It is emphasized that differently designed exemplary embodiments of an image acquisition assembly in the sense of the invention, as described above, can advantageously also be used in the endoscope.

FIG. 1 shows a distal end 12 of the shaft 11. The shaft 11 is shown transparent so that the image acquisition assembly 20 in the interior of the shaft 11 is visible. Two narrow and long and at least partially flexible circuit boards 24, 25 extend in the shaft 11 to the image acquisition assembly 20. Proximal ends (not shown in FIG. 1) of the circuit boards 24, 25 are connected to other assemblies by means of plug-in connectors, in particular at a proximal end (not shown) of the optical instrument 10. Electronic components (shown schematically in FIG. 1) for controlling or conditioning electrical power and for processing control signals and image signals can be provided on the circuit boards 24, 25. The circuit boards 24, 25 serve to supply electrical power and control signals to the image acquisition assembly and also to process image signals and transmit them to the proximal end of the optical instrument 10.

The image acquisition assembly 20 comprises a prism 30 having a first planar surface region that comprises a light entry surface 31. Arranged distally to the light entry surface 31 is an objective lens (not shown in FIG. 1) which produces an image of an object arranged outside the endoscope 10. The prism 30 further comprises a dichroic reflective layer or interface 32, the edge of which is visible in FIG. 1 as a line on the planar surface region of the prism 30 that comprises the light entry surface 31. The prism 30 further comprises two light exit surfaces, facing away from the viewer and therefore not visible in FIG. 1, which are formed by a second and a third planar surface region of the prism 30 and on which two image sensors 40, 50 are arranged.

The first image sensor 40 is mechanically and electrically connected to the distal end of the first circuit board 24 so as, via the first circuit board 24, to receive electrical power and control signals and to transmit image signals to a proximal end of the optical instrument 10. The second image sensor 50 is mechanically and electrically connected to the distal end of the second circuit board 25 so as, via the second circuit board 25, to receive electrical power and control signals and to transmit image signals to a proximal end of the optical instrument 10.

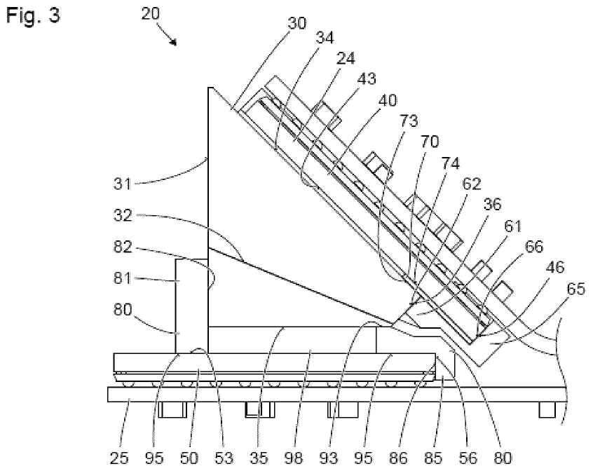

The prism 30 further comprises a fourth planar surface region, which in the illustration in FIG. 1 likewise faces away from the viewer. The fourth planar surface region faces away from the first planar surface region, which comprises the light entry surface 31, and does not have any optical function. As described with reference to FIG. 3, the fourth planar surface region forms a mechanical stop surface.

The prism 30 further comprises a fifth planar surface region 37 and a sixth planar surface region, which are parallel and face away from each other. In the illustration in FIG. 1, the fifth planar surface region 37 faces toward the viewer but is largely hidden. The fifth planar surface region 37 and the sixth planar surface region are orthogonal to the light entry surface 31 and to the two light exit surfaces.

The image acquisition assembly further comprises a first mount 60, which mechanically rigidly and preferably permanently connects the first image sensor 40 to the prism 30. The first mount 60 partially surrounds the prism 30 and is connected to the prism 30, for example by an adhesive (not shown in FIG. 1).

The image acquisition assembly further comprises a second mount 80, which mechanically rigidly and preferably permanently connects the second image sensor 50 to the prism 30. The second mount 80 partially surrounds the prism 30 and is connected to the prism 30, for example by an adhesive (not shown in FIG. 1).

In the exemplary embodiment of the image acquisition assembly 20 shown in FIGS. 1 to 7, the first mount 60 and the second mount 80 are in the form of separate components which are not connected to each other.

FIG. 2 contains a schematic and enlarged axonometric illustration of the image acquisition assembly 20 of FIG. 1. The viewing direction, from which the image acquisition assembly 20 is shown, corresponds to that of FIG. 1. Contours of the shaft 11 are not shown.

The first mount 60 comprises a first frame portion 61 (not visible in FIG. 2) and a plurality of symmetrically arranged second frame portions 63, which together partially surround the prism 30 on three sides. The first frame portion 61 (not visible in FIG. 2) of the first mount 60 bears against the fourth surface region 36 (not visible in the illustration in FIG. 2) of the prism 30, which faces away from the light entry surface 31 of the prism 30 (see FIG. 3). Alternatively, the first frame portion 61 (not visible in FIG. 2) may be arranged at a small distance opposite the fourth surface region 36 of the prism 30. The symmetrically arranged second frame portions 63 of the first mount 60 bear against the fifth planar surface region 37 of the prism 30 and against the sixth planar surface region of the prism 30 (not visible in the illustration of FIG. 2), which is parallel to and faces away from the fifth planar surface region. Alternatively, each of the second frame portions 63 of the first mount 60 may be located at a small distance opposite the fifth surface region 37 and the sixth surface region of the prism 30.

The first mount 60 further comprises a third frame portion 65 (not visible in the illustration in FIG. 2) and a plurality of fourth frame portions 67 arranged symmetrically in pairs, which partially surround the first image sensor 40 on three sides. The third frame portion 65 (not visible in FIG. 2) of the first mount 60 and the fourth frame portions 67 of the first mount 60, which are arranged symmetrically in pairs, bear against strip-shaped lateral edge surfaces 48 of the first image sensor 40. These strip-shaped lateral edge surfaces are in particular orthogonal to the light entry surface 43 of the first image sensor 40. Both the third frame portion 65 (not visible in FIG. 2) and the symmetrically arranged fourth frame portions 67 of the first mount 60 can each alternatively be located at a small distance opposite the associated strip-shaped lateral edge surface 46, 48 of the first image sensor 40.

The first image sensor 40 and the first mount 60 are permanently and mechanically rigidly connected in a materially bonded manner, in particular by means of an adhesive (not shown in FIG. 2). The first mount 60 and the prism 30 are permanently and mechanically rigidly connected in a materially bonded manner, in particular by means of an adhesive or solder (not shown in FIG. 2). Provided in the second frame portions 63 of the first mount 60 are through-openings 69, through which liquid adhesive or liquid solder can be applied.

The second mount 80 comprises a first frame portion 81 and a plurality of symmetrically arranged second frame portions 83, which partially surround the prism 30 on three sides. The first frame portion 81 of the second mount 80 bears against an edge region of the first planar surface region of the prism 30 that comprises the light entry surface 31. Alternatively, the first frame portion 81 is arranged at a small distance opposite the edge region of the first planar surface region of the prism 30. The symmetrically arranged second frame portions 83 of the second mount 80 bear against the fifth planar surface region 37 of the prism 30 and against the sixth planar surface region (not visible in the illustration of FIG. 2) of the prism 30, which is parallel to and faces away from the fifth planar surface region. Each of the second frame portions 83 of the second mount 80 may alternatively be located at a small distance opposite the fifth surface region 37 and the sixth surface region of the prism 30.

The second mount 80 further comprises a third frame portion 85 (not visible in the illustration in FIG. 2) and a plurality of fourth frame portions 87 arranged symmetrically in pairs, which together partially surround the second image sensor 50 on three sides. The third frame portion 85 (not visible in FIG. 2) and the symmetrically arranged fourth frame portions 87 of the second mount 80 bear against strip-shaped lateral edge surfaces 58 of the second image sensor 50. Both the third frame portion (not visible in FIG. 2) and the symmetrically arranged fourth frame portions 87 of the second mount 80 can each alternatively be located at a small distance opposite the associated strip-shaped lateral edge surface of the second image sensor 50.

The second image sensor 50 and the second mount 80 are permanently and mechanically rigidly connected in a materially bonded manner, in particular by means of a solder. The second mount 80 and the prism 30 are permanently and mechanically rigidly connected in a materially bonded manner, in particular by means of an adhesive or a solder. Provided in the second frame portions 83 of the second mount 80 are through-openings 89, through which liquid adhesive or liquid solder can be applied.

FIG. 3 contains a schematic illustration of a section through the image acquisition assembly 20 shown in FIGS. 1 and 2. The sectional plane of FIG. 3 is parallel to the planar surface regions 37 of the prism 30 (cf. FIGS. 1 and 2), orthogonal to the light entry surface 31, orthogonal to the dichroic reflective layer or interface 32, and orthogonal to the light exit surfaces 34, 35 of the prism. In particular, the sectional plane of FIG. 3 includes the optical axis of an objective lens (not shown in the figures). Some features which are not visible in the illustrations in FIGS. 1 and 2 are visible in the section in FIG. 3.

In the example of the image acquisition assembly according to the invention that is shown in FIG. 3, a beam-splitting prism 30 is provided, the light entry surface 31 and the first light exit surface 34 of which enclose an angle of 45 degrees, and the light entry surface 31 and the second light exit surface 35 of which enclose an angle of 90 degrees. The dichroic reflective layer or interface 32 encloses an angle of 22.5 degrees in each case with the first light exit surface 34 and with the second light exit surface 35. Of course, other geometries for the prism may also be provided. In the context of the invention, the technical design of the holding device can readily be transferred to such prisms.

The first frame portion 61 of the first mount 60 has a stop surface 62 which bears against the fourth planar surface region 36 of the prism 30 that adjoins the first light exit surface 34 and the second light exit surface 35 of the prism 30. The stop surface 62 on the first frame portion 61 of the first mount 60 and the fourth planar surface region 36 of the prism 30 are each orthogonal to the sectional plane of FIG. 3.

The third frame portion 65 of the first mount 60 has a stop surface 66 which bears against a first strip-shaped lateral edge surface 46 of the first image sensor 40. The stop surface 66 on the third frame portion 65 of the first mount 60 and the first strip-shaped lateral edge surface 46 of the first image sensor 40 are each orthogonal to the sectional plane of FIG. 3. The stop surface 62 and the stop surface 66 together form a stop structure of the first mount 60, comprising the stop surface 62 as a first stop surface and the stop surface 66 as a second stop surface of the mount 60. Here, the fourth planar surface region 36 of the prism 30 forms a first stop surface of the prism.

The first mount 60 is substantially U-shaped. The sectional plane of FIG. 3 intersects the first mount 60 only in the region of the first frame portion 61 and the third frame portion 65.

The first mount 60 comprises a film component 70. The film component 70 is formed of a planar film of constant thickness and consequently has two parallel surface regions 73, 74 facing away from each other. The film component 70 is U-shaped. The sectional plane of FIG. 3 intersects the film component 70 only in a region close to the first frame portion 61 and the third frame portion 65 of the first mount 60. The first planar surface region 73 of the film component 70 bears against an edge region of the first light exit surface 34 of the prism 30. The second planar surface region 74 of the film component bears against an edge region of the light entry surface 43 of the image sensor 40. The first planar surface region 73 forms a third stop surface and the second planar surface region 74 forms a fourth stop surface of the stop structure of the mount 60.

The film component 70 defines a first gap with a predetermined spacing and an exact parallelism between the first light exit surface 34 of the prism 30 and the light entry surface 43 of the first image sensor 40. As can be seen from FIG. 3, the first gap extends across most of the interior region of the prism 30. In particular, the gap extends across the projection of the light entry surface 31 onto the first light exit surface 34, so that any light beams incident into the prism can be reflected at an optical layer, formed by the gap, toward the interface 32. The gap serves to totally reflect the incident light.

The first frame portion 81 of the second mount 80 has a stop surface 82 which bears against an edge region of the first planar surface region that comprises the light entry surface 31 of the prism 30. The stop surface 82 on the first frame portion 81 of the second mount 80 and the first planar surface region of the prism 30 are each orthogonal to the sectional plane of FIG. 3.

The third frame portion 85 of the second mount 80 has a stop surface 86 which bears against a first strip-shaped lateral edge surface 56 of the second image sensor 50. The stop surface 86 on the third frame portion 85 of the second mount 80 and the first strip-shaped lateral edge surface 56 of the second image sensor 50 are each orthogonal to the sectional plane of FIG. 3. The stop surface 82 and the stop surface 86 together form a stop structure of the second mount 80, comprising the stop surface 82 as a first stop surface and the stop surface 86 as a second stop surface of the mount 80. The light entry surface 31 of the prism 30 can provide a second stop surface of the prism.

The second mount 80 has a first planar surface region 93 which bears against an edge region of the second light exit surface 35 of the prism 30, and a second planar surface region 95 which bears against an edge region of the light entry surface 53 of the second sensor 50. The second planar surface region 95 is parallel to the first planar surface region 93 and faces away therefrom. The second mount 80 and the second planar surface region 95 of the second mount have a circular topology and surround a circular or oval or rectangular window 98 that allows light to pass out of the prism 30 to the second image sensor 50. In the example shown, the first planar surface region 93 is C-shaped, thus surrounding the window 98 only largely, but not completely.

The parallel planar surface regions 93, 95 of the second mount 80 respectively form a stop surface for the prism 31 and the second image sensor 50. In particular, in this exemplary embodiment of the holding device, the first planar surface region 93 can form a third stop surface and the second planar surface region 95 can form a fourth stop surface of the stop structure of the mount 80. The surface regions 93, 95 define a predetermined spacing which forms a second gap arranged between the prism and the second image sensor. Furthermore, they establish an exact parallelism between the second light exit surface 35 of the prism 30 and the light entry surface 53 of the second image sensor 50.

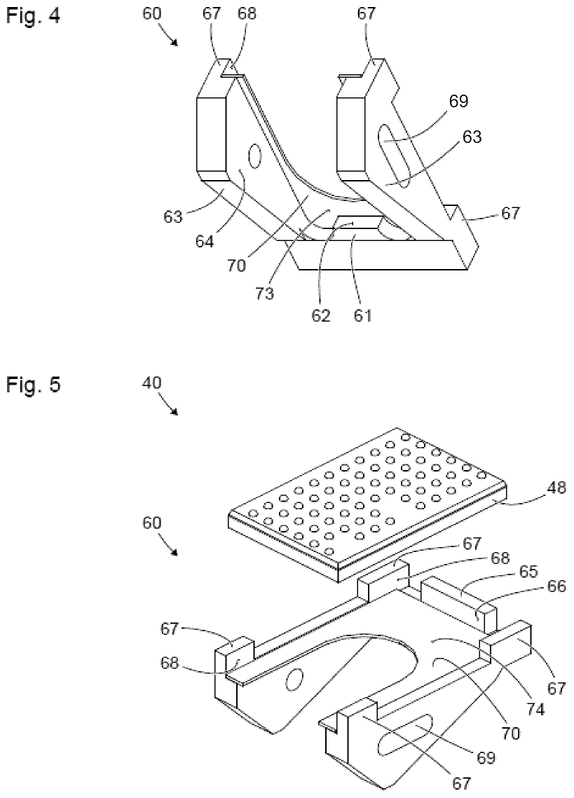

A light beam that is guided through the shaft 11 to the image acquisition assembly 20 and enters the prism 30 through the light entry surface 31 hits the light exit surface 34 at an angle of approximately 45 degrees. There, it is totally reflected at the optical layer formed by the first gap, so that at least approximately all of the light entering the prism 30 hits the dichroic reflective layer or interface 32. There, the light beam is split into a first sub-beam, for example comprising light in the visible range, and a second sub-beam, for example comprising light in the infrared range. The first sub-beam is directed from the interface to the first light exit surface 34 of the prism 30, and to the light entry surface 43 of the first image sensor 40, where it hits at an angle of approximately 90 degrees and thus can penetrate through the optical layer of the first gap into the image sensor 40. The second sub-beam is directed from the interface to the second light exit surface 35 of the prism 30, and to the light entry surface 53 of the second image sensor 50, where it hits at an angle of approximately 90 degrees and can penetrate through the optical layer of the second gap into the image sensor 50. To ensure this beam path through the image acquisition assembly 20, the mounts 60, 80 of the holding device are designed as shown in detail in FIG. 4 to 7. FIG. 4 contains a schematic axonometric illustration of the first mount 60 in a first view.

FIG. 4 shows the first planar surface region 73 of the film component 70, which is intended to bear against an edge region of the first light exit surface 34 of the prism 30 (cf. FIGS. 1 to 3). The stop surface 62 on the first frame portion 61 and the stop surface 64 on a second frame portion 63 of the first mount 60 can also be seen. The U-shaped arrangement of the first frame portion 61 and the second frame portions 63 of the first mount 60 can also be seen, the stop surfaces 62, 64 of which define, by way of a form fit, the arrangement and orientation of the first mount 60 relative to the prism 30. The fourth frame portions 67 and a stop surface 68 on one of the fourth frame portions 67 can also be seen.

FIG. 5 contains a schematic axonometric illustration of the first mount 60 in a second view. FIG. 5 also shows the first image sensor 40. The first image sensor 40 is shown at a distance from the first mount 60. However, in the assembled state of the image acquisition assembly, the first image sensor 40 is oriented relative to the first mount 60 in the same way as in the case of the image acquisition assembly 20 shown in FIGS. 1 to 3.

A second strip-shaped lateral edge surface 48 of the first image sensor 40 can be seen in FIG. 5. The second planar surface region 74 of the film component 70 can also be seen, which is intended to bear against an edge region of the light entry surface 43 of the first image sensor 40. The aforementioned U-shape of the film component 70 can also be seen. The stop surface 66 on the third frame portion 65 and stop surfaces 68 on fourth frame portions 67 of the first mount 60 can also be seen. The U-shaped arrangement of the third frame portion 65 and the fourth frame portions 67 of the first mount 60 can also be seen, the stop surfaces 66, 68 of which define, by way of a form fit, the arrangement and orientation of the first image sensor 40 relative to the first mount 60.

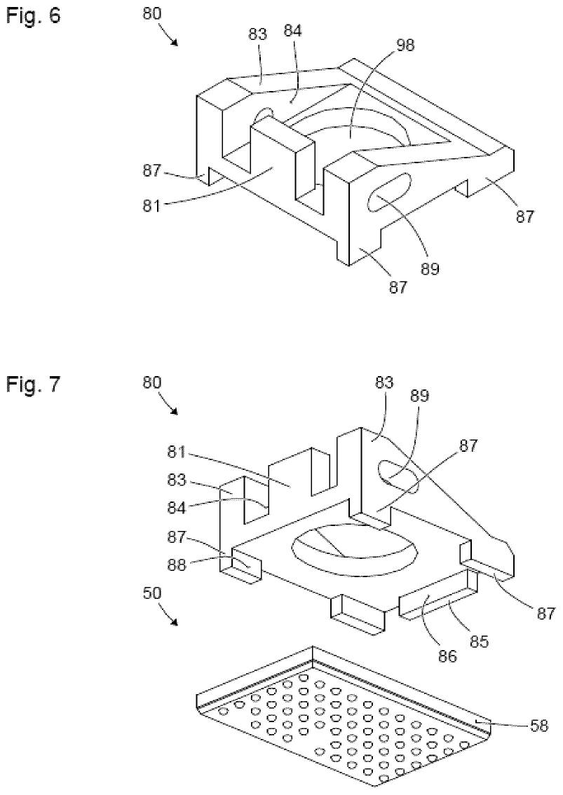

FIG. 6 contains a schematic axonometric illustration of the second mount 80 in a first view.

The aforementioned circular topology of the second mount 80 and the window 98 in the second mount can be seen in FIG. 6. The first planar surface region 93 of the second mount 80 can also be seen, which is intended to bear against an edge region of the second light exit surface 35 of the prism 30 (cf. FIGS. 1 to 3). The stop surface 84 on a second frame portion 83 of the second mount 80 can also be seen. The overall U-shaped arrangement of the first frame portion 81 and the second frame portions 83 of the second mount 80 can also be seen, the stop surfaces 84 of which define, by way of a form fit, the arrangement and orientation of the second mount 80 relative to the prism 30 (cf. FIGS. 1 to 3).

FIG. 7 contains a schematic axonometric illustration of the second mount 80 in a second view. FIG. 7 also shows the second image sensor 50. The second image sensor 50 is shown at a distance from the second mount 80. However, the second image sensor 50 is oriented relative to the second mount 80 in the same way as in the case of the image acquisition assembly 20 shown in FIGS. 1 to 3.

A second strip-shaped lateral edge surface 58 of the second image sensor 80 can be seen in FIG. 7. The second planar surface region 95 of the second mount 80 can also be seen, which is intended to bear against an edge region of the light entry surface 53 of the second image sensor 50 (cf. FIGS. 1 to 3). The stop surface 86 on the third frame portion 85 and stop surfaces 88 on fourth frame portions 87 of the second mount 80 can also be seen. The U-shaped arrangement of the third frame portion 85 and the fourth frame portions 87 of the second mount 80 can also be seen, the stop surfaces 86, 88 of which define, by way of a form fit, the arrangement and orientation of the second image sensor 50 relative to the second mount 80.

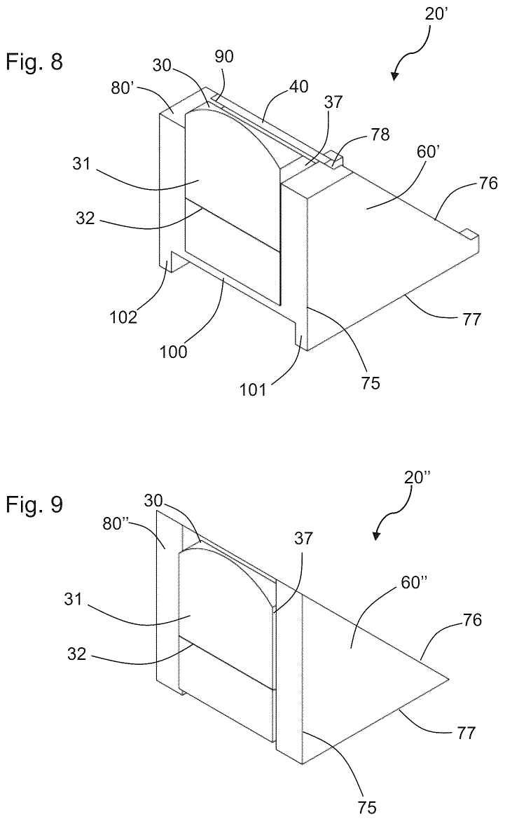

FIG. 8 shows another variant of an image acquisition assembly 20′ according to the invention, which again comprises a prism 30 as in the variant described above. The prism 30 comprises the light entry surface 31, the dichroic reflective layer or interface 32, and the two light exit surfaces, on each of which one of the image sensors 40, 50 is arranged. Furthermore, the prism 30 comprises two parallel, opposite side surfaces in the form of the fifth planar surface region 37 and the sixth planar surface region, as described above.

The image acquisition assembly 20′ further comprises a holding device comprising a first mount 60′ and a second mount 80′. The first mount 60′ and the second mount 80′ are rigidly connected to each other by a common base 100. In this variant of the image acquisition assembly 20′, the first mount 60′, the second mount 80′ and the base 100 form a one-piece holding device. However, they may also be connected, for example, by a welded or soldered connection, an adhesive connection, a crimped connection or another connection based on plastic deformation, and/or a screw connection.

The base 100 extends orthogonally between the parallel mounts 60′ and 80′. This means that the base 100 is perpendicular to opposite, inward-facing surfaces of the mounts 60′ and 80′. The mounts 60′ and 80′ are arranged symmetrically in relation to a central plane extending centrally through the base 100. The first mount 60′, the second mount 80′ and the base 100 form a U-shaped structure for receiving the prism 30 in the space between the mounts 60′ and 80′. The first mount 60′ and the second mount 80′ are designed as side pieces, the edge geometry of which substantially corresponds to the geometry of the light entry and exit surfaces of the prism 30. This means that a distal edge 75, a proximal edge 76 and an edge 77 shown at the bottom of the mounts in the drawing are at the same angles to one another as the light entry and exit surfaces 31, 34 and 35 of the prism 30, as shown in FIG. 8 by way of example for the mount 60′. The inward-facing inner surfaces of the mounts 60′ and 80′ each form a stop surface for bearing against the opposite side surfaces of the prism 30. The fifth planar surface region 37 forms a stop surface of the prism for bearing against the inner surface of the first mount 60′, and the sixth planar surface region forms a stop surface of the prism for bearing against the inner surface of the second mount 80′.

On the side of the base 100 located opposite the U-shaped receptacle for the prism 30, opposite frame portions 101 and 102 project beyond the base 100 at the lower edges 77 of the first mount 60′ and the second mount 80′. The frame portion 101 on the first mount 60′ forms a first stop surface and the frame portion 102 on the second mount 80′ forms a second stop surface for an image sensor (not shown), which is provided on the second light exit surface 35 of the prism 30. The frame portions 101 and 102 have approximately the same function as the fourth frame portions 87 from the variant of the image acquisition assembly 20 shown in FIGS. 1 to 7. The base 100 may optionally have a further frame portion formed at a proximal end region of the base between the frame portions 101 and 102. For example, the further frame portion is designed analogously to the frame portion 85 of the variant shown in FIGS. 1 to 7. The image sensor can rest on the surface of the base 100. The base 100 is advantageously designed with a window, for example in the form of an annular topology, as described for the second mount 80 of the variant shown in FIGS. 1 to 7. Alternatively, the base may also have a window in the form of a U-shaped or C-shaped cutout, analogous to the U-shaped or C-shaped design of the first mount 60 of the variant of FIGS. 1 to 7. A rectangular cutout as a window or in the form of a slot is also conceivable. In principle, there should be a sufficiently large window that allows light beams to pass through from the second light exit surface 35 to the image sensor and yet forms a stable base for the holding device.

The first mount 60′ has a step 78 along its proximal edge 76. The second mount 80′ has a step 90 along its proximal edge. In the region of the steps 78 and 90, the thickness of the respective mount is reduced and the distance between the opposite inner surfaces of the first and second mounts 60′, 80′ is increased. The step 78 on the first mount 60′ forms a first stop surface for a further image sensor, for example for bearing against a strip-shaped lateral edge surface of the image sensor, for instance against the strip-shaped lateral edge surface 48 of the image sensor 40 from FIGS. 1 to 7. The step 90 on the second mount 80′forms a second stop surface for the further image sensor, for instance against the strip-shaped lateral edge surface 46 of the image sensor 40. The steps 78 and 90 form frame portions for receiving the image sensor on the first light exit surface 34, for example analogous to the fourth frame portions 67 of the variant from FIGS. 1 to 7. Since the geometry of the mounts 60′ and 80′ corresponds to the geometry of the prism, the surface of the image sensor extends parallel to the light exit surface 34 of the prism. A gap may remain as an optical layer between the image sensor and the prism, as described above. To this end, for example, an inwardly projecting protrusion may be provided adjacent to the steps 78 and 90 along the proximal edges 76 on the mounts 60′ and 80′. A film component may also be provided, such as the film component 70 of the variant from FIGS. 4 and 5. As an alternative or in addition, a frame portion projecting from the base 100 in the direction of the prism may be provided, which prevents the prism from butting against the image sensor.

In the assembled state of the image acquisition assembly 20′, the first mount 60′ and the second mount 80′ each come to lie against the side surfaces, such as the fifth planar surface region 37, of the prism 30. The base 100 comes to lie against the second light exit surface 35 of the prism 30. The image sensor, for instance the first image sensor 40, comes to lie between the steps 78 and 90 and bears against the surfaces thereof. The further image sensor, for instance the second image sensor 50, comes to lie between the frame portions 101 and 102 and adjacent to the base 100. Owing to the design of the holding device of the image acquisition assembly 20′, the prism and the two image sensors are in a defined position relative to one another. The mounts 60′ and 80′ may have through-openings, analogous to the through-openings 69 and 89, in order to fix the prism in the holding device.

In summary, the first mount 60′ has a stop for bearing against the stop surface of the prism 30 in the form of the side surface 37 and for bearing against stop surfaces of the image sensors, wherein the stop comprises stop surfaces in the form of the inner surface of the mount 60′, the frame portion 101 and the step 78. The second mount 80′ has a stop for bearing against the stop surface of the prism 30 in the form of the opposite side surface and for bearing against stop surfaces of the image sensors, wherein the stop comprises stop surfaces in the form of the inner surface of the mount 80′, the frame portion 102 and the step 90. As mentioned above, further stop surfaces may be provided on the mounts 60′ and 80′ and the base 100, which aid the positioning of the image sensors and the prism relative to one another. Furthermore, further features described in connection with the variant shown in FIGS. 1 to 7 can advantageously be adopted for the image acquisition assembly 20′.

The holding device of the image acquisition assembly 20′ forms a kind of overall mount for the prism and the image sensors. Such an overall mount allows a high degree of freedom in adjustment, a reduction in required tolerances, and a reduction in accumulated tolerances.

FIG. 9 shows yet another variant of an image acquisition assembly 20″ according to the invention, which again comprises a prism 30 as in the previously described variants.

The image acquisition assembly 20″ comprises a holding device with a first mount 60″ and a second mount 80″, which are designed as isosceles right-angled wedge-shaped pieces. The first mount 60″ and the second mount 80″ are in the form of separate components. The wedge-shaped pieces are placed against the parallel, opposite side surfaces in the form of the fifth planar surface region 37 and the sixth planar surface region such that their distal edge 75 extends parallel to the light entry surface 31, their proximal edge 76 extends parallel to the first light exit surface 34, and their lower edge 77 extends parallel to the light exit surface 35. For example, the proximal edge 76 and the lower edge 77 are arranged flush with the prism surfaces. Subsequently, a first image sensor is attached to the edge surfaces of the proximal edges 76 and a second image sensor is attached to the edge surfaces of the lower edges 77. By way of example, the image sensors are glued. The image sensors are attached one after the other to the wedge-shaped pieces. During this, either the prism or the wedge-shaped pieces can be moved in order to set specified distances.

In this variant of the image acquisition assembly 20″, the holding device is realized by fixing the wedge-shaped pieces in the form of the first mount 60″ and the second mount 80″ to the image sensors in order to obtain a mechanically rigid connection of the image sensors.

In this variant, the first mount 60″ and the second mount 80″ each have a stop in the form of the stop surfaces formed by the surfaces of the proximal edges 76 for bearing against a stop surface of the first image sensor that is formed, for example, by outer edge regions of the image sensor. Furthermore, the first mount 60″ and the second mount 80″ each have a stop in the form of the stop surfaces formed by the surfaces of the lower edges 77 for bearing against a stop surface of the second image sensor that is also formed, for example, by outer edge regions of the image sensor. Furthermore, the first mount 60″ and the second mount 80″ each have a stop surface for bearing against the side surface 36 of the prism 30, these stop surfaces being formed by the triangular surfaces of the wedge-shaped pieces, as shown in FIG. 9.

This variant of the image acquisition assembly 20″ allows for a simple and cost-effective design of the components of the holding device. Only sufficient accuracy regarding the angles of the wedge-shaped pieces is required. Since the two wedge-shaped pieces are attached independently of each other to the side surfaces of the prism, this distance between the wedge-shaped pieces does not require any tolerance. Designing the wedge-shaped pieces without guide surfaces for the image sensors brings the advantage of a high degree of freedom. Tolerances can be compensated by means of a precise optical orientation of the image sensors.

Alternatively, small steps or protrusions can be provided on the wedge-shaped pieces as a stop for the prism and/or the image sensors, said steps or protrusions projecting in the direction of the prism and/or the image sensors. The steps or protrusions may project only a small distance from the wedge surface of the wedge-shaped pieces that bears laterally against the prism, so that only an edge region of the prism or image sensors is covered.

The wedge-shaped pieces may also be designed, for example, as a combination of the second frame portion 63 of the first mount 60 and the second frame portion 83 of the second mount 80. In this exemplary embodiment, there is no need to connect the opposite frame portions, such as the first frame portion 61 in the case of the mount 60 or the first frame portion 81 in the case of the mount 80.

Although the invention is illustrated and described in detail by means of the figures and the associated description on the basis of exemplary embodiments of an image acquisition assembly according to the invention, these illustrations and the detailed descriptions thereof are to be understood as illustrative and exemplary and not as limiting the invention. It will be understood that a person skilled in the art may make changes and modifications without departing from the scope of the technical features of the image acquisition assembly that define the invention. In particular, the present invention covers other exemplary embodiments involving combinations of features that may differ from the exemplary embodiment shown in the figures. By way of example, the invention may also be realized as follows:

The first mount and the second mount are at least either directly mechanically rigidly connected or provided as one piece. To this end, for example, the first frame portion 61 of the first mount 60 may be connected to the second mount 80 in the region of the third frame portion 85. Consequently, the second mount 80 does not have to bear against the prism 30 and the second sensor 50. It may also bear only against the second sensor 50. Furthermore, the image acquisition assembly may be designed in the manner explained in connection with FIG. 8.

The stop of the first mount 60 may have a common stop surface for bearing against the first stop surface 36 of the prism and against the stop surface 46 of the first image sensor 40, and/or the stop of the second mount 80 may have a common stop surface for bearing against a stop surface of the prism and against a stop surface of the second image sensor 50, for example the first stop surface 82 or the second stop surface 86.

The first mount and the second mount may be designed as separate components, each of which is attached to the side of the prism, for example to the fifth surface region 37 shown in FIG. 2 and the complementary opposite sixth surface region. To this end, the mounts may be designed as in the example of FIG. 8, but without a base 100. Furthermore, the mounts may be designed as wedge-shaped pieces that correspond to the lateral surface geometry of the prism, i.e., analogous to the shape of the fifth and sixth surface regions of the prism, as in the variant of the image acquisition assembly of FIG. 9 for example.

The image acquisition assembly may comprise more than two image sensors. Two or more image sensors may be arranged on the same light exit surface of the prism. In this case, two or more image sensors may be arranged on one mount of the holding device, wherein the stop of the mount advantageously has one stop surface for each image sensor. The prism may also have more than two light exit surfaces. In this case, the holding device has, for each light exit surface, a mount which is adapted to the geometry of the prism or the light exit surfaces thereof, in a manner analogous to the first mount 60 and the second mount 80.

The exemplary embodiments listed comprise further features as shown in the example illustrated in the figures, even if they are shown there in connection with other features. For a person skilled in the art, it is readily apparent from the description which features from the figures can be transferred to the exemplary embodiments listed.

### LIST OF REFERENCE SIGNS

10 optical instrument
11 shaft of the optical instrument 10
12 distal end of the shaft 12 of the optical instrument 10
20, 20′ 20″ image acquisition assembly of the optical instrument 10
24 first at least partially flexible circuit board
25 second at least partially flexible circuit board
30 prism of the image acquisition assembly 20
31 light entry surface of the prism 30
32 dichroic reflective layer or interface of the prism 30
34 first light exit surface of the prism 30
35 second light exit surface of the prism 30
36 fourth planar surface region of the prism 30, adjoining the light exit surfaces 34, 35
37 fifth planar surface region of the prism 30, adjoining the light exit surfaces 34, 35
40 first image sensor of the image acquisition assembly 20
43 light entry surface of the first image sensor 40
46 first strip-shaped lateral edge surface of the first image sensor 40
48 second strip-shaped lateral edge surface of the first image sensor 40
50 second image sensor of the image acquisition assembly 20
53 light entry surface of the second image sensor 50
56 first strip-shaped lateral edge surface of the second image sensor 50
58 second strip-shaped lateral edge surface of the second image sensor 50
60, 60′, 60″ first mount for mechanically rigidly mounting the first image sensor 40 on the first light exit surface 34 of the prism 30
61 first frame portion of the first mount 60
62 stop surface on the first frame portion 61 of the first mount 60, for bearing against or being arranged opposite the fourth planar surface region 36 of the prism 30
63 second frame portion of the first mount 60
64 stop surface on the second frame portion 63 of the first mount 60, for bearing against or being arranged opposite the sixth planar surface region of the prism 30
65 third frame portion of the first mount 60
66 stop surface on the third frame portion 65 of the first mount 60, for bearing against or being arranged opposite the first strip-shaped lateral edge surface 46 of the first image sensor 40
67 fourth frame portion of the first mount 60
68 stop surface on the fourth frame portion 67 of the first mount 60, for bearing against or being arranged opposite the second strip-shaped lateral edge surface 48 of the first image sensor 40
69 through-opening in the second frame portion 63 of the first mount 60
70 Film Component
73 first planar surface region of the film component 70, bearing against an edge region of the first light exit surface 34 of the prism 30
74 second planar surface region of the film component 70, bearing against an edge region of the light entry surface 43 of the first image sensor 40
75 distal edge of the first mount 60′
76 proximal edge of the first mount 60′
77 edge of the mount 60′ located at the bottom in the drawing
80, 80′, 80″ second mount for mechanically rigidly mounting the second image sensor 50 on the second light exit surface 35 of the prism 30
81 first frame portion of the second mount 80
82 stop surface on the first frame portion 81 of the second mount 80, for bearing against an edge region of the light entry surface 31 of the prism 30
83 second frame portion of the second mount 80
84 surface region on the second frame portion 83 of the second mount 80, for being arranged opposite the second planar surface region 37 of the prism 30 that adjoins the light exit surfaces 34, 35
85 Third Frame Portion of the Second Mount 80
86 stop surface on the third frame portion 85 of the second mount 80, for bearing against the first strip-shaped edge surface 56 of the second image sensor 50
87 fourth frame portion of the second mount 80
88 surface region on the fourth frame portion 87 of the second mount 80, for being arranged opposite the second strip-shaped edge surface 58 of the second image sensor 50
89 through-opening in the second frame portion 83 of the second mount 80
93 first planar surface region of the second mount 80 bearing against an edge region of the second light exit surface 35 of the prism 30
95 planar surface region of the second mount 80 bearing against an edge region of the light entry surface 53 of the second image sensor 50
96 window in the second mount 80
100 base between the first mount 60′and the second mount 80′
101 frame portion of the first mount 60′
102 frame portion of the second mount 80′

1. An image acquisition assembly, comprising:
a beam-splitting prism having a light entry surface, a dichroic reflective layer or interface, at least a first light exit surface and a second light exit surface; at least two image sensors, wherein
a first image sensor is arranged on the first light exit surface of the prism and a second image sensor is arranged on the second light exit surface of the prism ; and
a holding device which comprises a first mount and a second mount for mechanically rigidly connecting the first image sensor and the second image sensor to the prism, wherein the first mount has at least one stop for bearing against a first stop surface of the prism and for bearing against a stop surface on the first image sensor, and
the second mount has at least one stop for bearing against a second stop surface of the prism and/or for bearing against a stop surface on the second image sensor.

2. The image acquisition assembly of claim 1, wherein the first mount and the second mount are two separate components.

3. The image acquisition assembly of claim 1, wherein the first mount and the second mount are mechanically rigidly connected or provided as one piece.

4. The image acquisition assembly of claim 1, wherein either
a stop surface of the first mount is orthogonal to the first light exit surface of the prism or is orthogonal to a light entry surface of the first image sensor, or
a stop surface of the second mount is orthogonal to the second light exit surface of the prism or is orthogonal to a light entry surface of the second image sensor.

5. The image acquisition assembly of claim 1, wherein the stop of the first mount has a common stop surface for bearing against the first stop surface of the prism and against the stop surface of the first image sensor, or
the stop of the second mount has a common stop surface for bearing against the second stop surface of the prism or against the stop surface of the second image sensor.

6. The image acquisition assembly of claim 4, wherein the stop of the first mount has a first stop surface for bearing against the first stop surface of the prism and a second stop surface for bearing against the stop surface on the first image sensor, or
the stop of the second mount has a first stop surface for bearing against the second stop surface of the prism and a second stop surface for bearing against the stop surface on the second image sensor.

7. The image acquisition assembly of claim 6, wherein at least either
the first stop surface of the first mount is orthogonal to the intended orientation of the first light exit surface of the prism, or
the second stop surface of the first mount is orthogonal to the intended orientation of a light entry surface of the first image sensor.

8. The image acquisition assembly of claim 6, wherein at least either
the first stop surface of the second mount is orthogonal to the intended orientation of the second light exit surface of the prism, or
the second stop surface of the second mount is orthogonal to the intended orientation of a light entry surface of the second image sensor.

9. The image acquisition assembly of claim 1, wherein
the stop of the first mount is intended to bear against a surface region of the prism that faces away from the light entry surface of the prism.

10. The image acquisition assembly of claim 1, wherein
the stop of the second mount is configured to bear at least partially against a surface region of the prism that comprises the light entry surface of the prism.

11. The image acquisition assembly of claim 1, wherein
the stop of the first mount has a stop surface for bearing against an edge region of the first light exit surface of the prism and a stop surface for bearing against an edge region of a light entry surface of the first image sensor.

12. The image acquisition assembly of claim 1, wherein
the stop of the second mount has a stop surface for bearing against an edge region of the second light exit surface of the prism and a stop surface for bearing against an edge region of a light entry surface of the second image sensor.

13. The image acquisition assembly according to claim 11, wherein
the first mount or the second mount comprises a film component having a first planar surface region and a second planar surface region,
the first planar surface region and the second planar surface region of the film component are parallel and face away from each other,
wherein the first planar surface region of the film component forms the stop surface for bearing against an edge region of the first light exit surface of the prism, and
the second planar surface region of the film component forms the stop surface for bearing against an edge region of a light entry surface of the first image sensor.

14. The image acquisition assembly of claim 11, wherein at least either
the stop surface for bearing against an edge region of the first light exit surface of the prism and the stop surface for bearing against an edge region of a light entry surface of the first image sensor of the first mount or the second mount are each U-shaped or C-shaped, or
the stop surface for bearing against an edge region of the second light exit surface of the prism and the stop surface for bearing against an edge region of a light entry surface of the second image sensor of the first mount or the second mount are each U-shaped or C-shaped.

15. The image acquisition assembly of claim 11, wherein at least either the stop surface for bearing against an edge region of the first light exit surface of the prism and the stop surface for bearing against an edge region of a light entry surface of the first image sensor of the first mount or the stop surface for bearing against an edge region of the second light exit surface of the prism and the stop surface for bearing against an edge region of a light entry surface of the second image sensor of the second mount are each annular.

16. The image acquisition assembly of claim 1, wherein the dichroic reflective layer or interface of the beam-splitting prism is arranged parallel to an angle bisector plane of the first light exit surface and the second light exit surface,
the first mount is U-shaped or C-shaped with an open side oriented toward the light entry surface of the prism, and the second mount has an annular topology.

17. The image acquisition assembly of claim 1, wherein at least either
the first mount comprises at least three frame portions which are each arranged opposite one of three different planar or substantially planar surface regions of the prism that adjoin the first light exit surface of the prism, or
the second mount comprises at least three frame portions which are each arranged opposite one of three different planar or substantially planar surface regions of the prism that adjoin the second light exit surface of the prism.

18. The image acquisition assembly of claim 1, further comprising:
at least one through-opening in the first mount or the second mount for applying adhesive or solder in order to connect the first mount to the prism or the second mount to the prism in a materially bonded manner.

19. A holding device for an image acquisition assembly comprising a beam-splitting prism and at least two image sensors, for mechanically rigidly holding a first image sensor on a first light exit surface of the prism and a second image sensor on a second light exit surface of the prism, said holding device comprising: a first mount for mechanically rigidly connecting the first image sensor to the prism, said first mount having at least one stop for bearing against a first stop surface of the prism and for bearing against a stop surface on the first image sensor, and
a second mount for mechanically rigidly connecting the second image sensor to the prism, said second mount having at least one stop for bearing against a second stop surface of the prism or for bearing against a stop surface on the second image sensor.

20. An optical instrument, comprising:
a shaft having a distal end, and
an image acquisition assembly arranged at the distal end of the shaft, the image acquisition assembly comprising
a beam-splitting prism having a light entry surface, a dichroic reflective layer or interface, at least a first light exit surface and a second light exit surface; at least two image sensors, wherein
a first image sensor is arranged on the first light exit surface of the prism and a second image sensor is arranged on the second light exit surface of the prism ; and
a holding device which comprises a first mount and a second mount for mechanically rigidly connecting the first image sensor and the second image sensor to the prism,
wherein the first mount has at least one stop for bearing against a first stop surface of the prism and for bearing against a stop surface on the first image sensor, and
the second mount has at least one stop for bearing against a second stop surface of the prism and/or for bearing against a stop surface on the second image sensor.
